(19) United States
(12) Patent Application Publication
(10) Pub. No.: US 2009/0291092 A1
(43) Pub. Date: Nov. 26, 2009

(54) TREATMENT OF MITOCHONDRIAL DISEASES WITH AN ERYTHROPOIETIN MIMETIC

(76) Inventors: Guy M. MILLER, Monte Sereno, CA (US); William D. SHRADER, Belmont, CA (US); Viktoria KHEIFETS, Mt. View, CA (US)

Correspondence Address: MORRISON & FOERSTER LLP, 755 PAGE MILL RD, PALO ALTO, CA, 94304-1018, US

(21) Appl. No.: 12/469,475

(22) Filed: May 20, 2009

Related U.S. Application Data
(60) Provisional application No. 61/128,626, filed on May 22, 2008.

(51) Int. Cl. A61K 39/395; A61K 38/02

(52) U.S. Cl. 424/178.1; 514/2

(57) ABSTRACT

Methods of treating mitochondrial disorders that are not respiratory chain disorders using compositions comprising EPO mimetic compounds or compounds capable of increasing endogenous EPO levels or stimulating erythropoiesis are disclosed. Methods of treating Friedreich's ataxia, Leigh's syndrome, or other disorders by increasing the expression of frataxin with an EPO mimetic compound or a compound capable of increasing endogenous EPO levels or stimulating erythropoiesis are also disclosed.

### CROSS-REFERENCE TO RELATED APPLICATIONS

This application claims priority benefit of U.S. Provisional Patent Application No. 61/128,626, filed May 22, 2008. The entire content of that application is hereby incorporated by reference herein in its entirety.

### TECHNICAL FIELD

The present invention discloses methods of treating mitochondrial disorders that are not respiratory chain disorders and for the treatment or prevention of diseases associated therewith, using at least one erythropoietin (EPO) mimetic composition in a subject in need of such treatment. The present invention also discloses methods for the treatment of mitochondrial disorders, and for the treatment or prevention of diseases associated therewith using at least one composition that is capable of increasing endogenous EPO levels, thus stimulating erythropoiesis, in a subject suffering from a mitochondrial disease. Particularly, the present invention discloses methods of treating Friedreich's Ataxia or Leigh's syndrome using at least one EPO mimetic composition or at least one compound capable of increasing endogenous EPO.

### BACKGROUND

Mitochondria are organelles in eukaryotic cells, popularly referred to as the “powerhouse” of the cell. The molecule adenosine triphosphate (ATP) functions as an energy “currency” or energy carrier in the cell, and eukaryotic cells derive the majority of their ATP from biochemical processes carried out by mitochondria. These biochemical processes include the citric acid cycle (the tricarboxylic acid cycle, or Krebs cycle), which generates reduced nicotinamide adenine dinucleotide (NADH+H⁺) from oxidized nicotinamide adenine dinucleotide (NAD⁺), and oxidative phosphorylation, during which NADH+H⁺ is oxidized back to NAD⁺. (The citric acid cycle also reduces flavin adenine dinucleotide, or FAD, to FADH₂; FADH₂ also participates in oxidative phosphorylation.)

In addition to their role in energy (ATP) production, mitochondria are involved in many processes that include synthesis of heme groups, steroids, amino acids, iron-sulfur cluster synthesis, cellular calcium buffering, mitochondria mediated apoptosis, and the mitochondrial stress response.

Mitochondrial dysfunction contributes to various disease states. Some mitochondrial diseases are due to mutations or deletions in the mitochondrial genome. If a threshold proportion of mitochondria in the cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved. Mitochondrial diseases encompass a broad range of phenotypes ranging from neuro-metabolic diseases to certain cancers. Clinical manifestations range from a single affected tissue to multi-organ disorders. Symptom onset can occur at essentially any age and in many cases, progression can be very slow and extend over decades.

In general, an organ's reliance on oxidative phosphorylation for proper functioning determines the likelihood that symptoms will occur in that organ. Consequently, the central nervous system is the most vulnerable organ to mitochondrial disease. Clinical manifestations are diverse and include for example mental retardation, dementia, leukoencephalopathy, psychiatric symptoms, epilepsy, ataxia, dystonia, vision loss, and hearing loss. The most common manifestations in other organs are cardiac dysfunction, muscle dysfunction, endocrinopathy, and hepatopathy. In pediatric patients, developmental delay and failure to thrive are common features. Growth failure unrelated to growth hormone secretion frequently occurs.

One such disease is Friedreich's ataxia (FRDA or FA). Friedreich's ataxia is an autosomal recessive neurodegenerative and cardiodegenerative disorder caused by decreased levels of the protein frataxin. Frataxin is important for the assembly of iron-sulfur clusters in mitochondrial respiratory-chain complexes. Estimates of the prevalence of FRDA in the United States range from 1 in every 22,000-29,000 people (see World-Wide-Web.nlm.nih.gov/medlineplus/ency/article/001411.htm) to 1 in 50,000 people (see World-Wide-Web.umc-cares.org/health_info/ADAM/Articles/001411.asp). The disease causes the progressive loss of voluntary motor coordination (ataxia) and cardiac complications. Symptoms typically begin in childhood, and the disease progressively worsens as the patient grows older; patients eventually become wheelchair-bound due to motor disabilities.

Friedreich's ataxia is caused by a GAA-trinucleotide expansion in the frataxin gene located on chromosome locus 9q13, resulting in a reduced expression of frataxin, a small mitochondrial protein (Campuzano et al., Hum. Mol. Genet. (1997); 6, 1771-1780). Due to the mitochondrial localization of frataxin, the neurological and cardiological degenerations observed in FRDA are thought to be the result of a mitochondrial defect (Tan et al., Hum. Mol. Genet. (2001), 19, 2099-2107). The exact physiological function of frataxin is unknown, but it may be involved in mitochondrial iron homeostasis and/or assembly of iron-sulfur (FeS) proteins and heme synthesis. Intra-mitochondrial iron accumulation has been postulated to initiate the production of hydroxyl radicals by Fenton chemistry, leading to inactivation of FeS enzymes, lipid peroxidation and damage to nucleic acids, proteins and finally resulting in cell death.

There is some debate whether mitochondrial iron accumulation within mitochondria is the result or the cause of the oxidative stress which is responsible for mitochondrial damage. Studies with conditional knockout mouse models and FRDA-patient cells indicate that deficiencies in FeS enzymes precede iron accumulation (Puccio et al., Nat. Genet. (2001) 27, 181-186). Clinically there is an intra-mitochondrial iron accumulation in heart, liver, nervous system and spleen of FRDA-patients, as well as a reduction of mitochondrial DNA, the FeS cluster-containing subunits of the mitochondrial electron transport chain (complex I-III) and of the enzyme aconitase (Bradley, Hum. Mol. Genet. (2000) 9; 275-283). The presence of increased levels of soluble transferrin receptor as indicator for cytosolic iron deficiency is controversial but in general FRDA-patients have normal serum iron and ferritin concentrations. Frataxin is implicated to be necessary for normal heme biosynthesis, but there are no reports that FRDA is commonly associated with anemia. Additional diseases with mitochondrial iron accumulation, including myelodysplastic syndromes and sideroblastic anemia also result in mitochondrial damage.

Leigh's syndrome is a rare inherited neuro-metabolic disorder characterized by degeneration of the central nervous system. Leigh's syndrome can be caused by mutations in mitochondrial DNA or by deficiencies of pyruvate dehydrogenase. Symptoms of Leigh's syndrome usually begin between the ages of 3 months to 2 years and progress rapidly. In most children, the first signs may be poor sucking ability and loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function. Heart problems may also occur. In rare cases, Leigh's syndrome can begin during late adolescence or early adulthood and progress more slowly.

In addition to congenital disorders involving inherited defective mitochondria, acquired mitochondrial dysfunction contributes to diseases, particularly neurodegenerative disorders associated with aging. Mitochondrial dysfunction is important in the pathogenesis of many common diseases including Parkinson's, Alzheimer's, amyotrophic lateral sclerosis (ALS) and Huntington's. The incidence of somatic mutations in mitochondrial DNA rises exponentially with age. Mitochondrial dysfunction is also implicated in excitotoxic neuronal injury and cerebrovascular accidents such as that associated with seizures, stroke and ischemia.

Very few treatments are available for patients suffering from these diseases. Recently, the compound idebenone has been proposed for treatment of Friedreich's ataxia. While the clinical effects of idebenone have been relatively modest, the complications of mitochondrial diseases can be so severe that even marginally useful therapies are preferable to the untreated course of the disease. Another compound, MitoQ, has been proposed for treating mitochondrial disorders (see U.S. Pat. No. 7,179,928); clinical results for MitoQ have not yet been reported.

Methods of treatment of a respiratory chain disorder, comprising administering a therapeutically effective amount of a composition comprising one or more molecules having erythropoietin activity, selected from EPO, or a biosimilar, a variant, a mutant or a mimetic thereof have been disclosed in co-owned PCT publication WO 2008/086025 filed Jan. 9, 2008.

Administration of human erythropoietin (EPO) or a derivative thereof having the biological activity of human erythropoietin of increasing the expression of frataxin, for the production of a pharmaceutical preparation for the treatment of Friedreich's ataxia or for the treatment or prevention of a disease associated therewith has been disclosed in PCT publication WO 2006/050819. However, it is well known in the art that epoetin alpha may cause several side effects such as an increase in blood pressure, chest pain, swelling due to retention of fluid, fast heart beat, headache, increase in number and concentration of circulating red blood cells, seizures, shortness of breath, skin rash, pain in joints, rapid gain weight, swelling of feet or joints, diarrhea, nausea, fatigue, or flu-like syndrome after each dose.

Nowhere is described the treatment of a mitochondrial disease that is not a respiratory chain disorder using an EPO mimetic or a compound that is capable of increasing endogenous EPO or stimulating erythropoiesis. Similarly nobody has disclosed the treatment of Friedreich's ataxia or Leigh's syndrome using an EPO mimetic or a compound that is capable of increasing endogenous EPO or stimulating erythropoiesis.

It is therefore an object of the present invention to provide a superior pharmaceutical preparation for the treatment of mitochondrial diseases that are not respiratory chain diseases, and for the treatment of Friedreich's ataxia or Leigh's syndrome, and for the treatment or prevention of a disease associated therewith, by administration to a patient with such a disease a composition of one or more compounds that are EPO mimetic or capable of increasing endogenous EPO or stimulating erythropoiesis.

### DISCLOSURE OF THE INVENTION

The invention embraces methods of treating mitochondrial disorders that are not a respiratory chain disorder, comprising administering a therapeutically effective amount of a composition comprising one or more EPO mimetic molecules or molecules capable of increasing the endogenous EPO or stimulating erythropoiesis, to an individual with a mitochondrial disorder that is not a respiratory chain disorder.

The invention embraces the use of one or more EPO mimetic molecules having the biological activity of increasing the expression of frataxin in a superior way than rhuEPO for the treatment of Friedreich's ataxia or Leigh's syndrome. In some embodiments the expression of frataxin by the EPO mimetic is increased by about, or by at least about, 50, 60, 70, 75, 80, 90, 100, 110, 120, 130, 140, 150, 160, 170, 180, 190, 200, 210, 220, 230, 240, 250, 260, 270, 280, 290, or 300 per cent. In some embodiments the expression of frataxin by the EPO mimetic in Friedreich's ataxia fibroblasts is at least two times greater than the expression of frataxin by EPO-beta. In some embodiments, the expression of frataxin by the EPO mimetic increases by about 50-300%, about 75%-250%, or particularly about 100-200%.

In one embodiment, the one or more EPO mimetic molecules comprise a protein or peptide mimetic of EPO or a small molecule mimetic of EPO. In a particular embodiment, the EPO mimetic is a protein or a peptide. In another embodiment, the EPO mimetic is a small molecule.

In one embodiment, the invention embraces methods of treating a mitochondrial disorder that is not a respiratory chain disorder, comprising administering, to an individual having a mitochondrial disorder that is not a respiratory chain disorder, a therapeutically effective amount of a composition comprising one or more EPO mimetic hinge core mimetibody polypeptides or specified fragments or variants thereof, including isolated nucleic acids that encode at least one EPO mimetic hinge core mimetibody or specific fragments or variants, or vectors that encode at least one EPO mimetic hinge core mimetibody or specific fragments or variants. The at least one EPO mimetic hinge core mimetibody or specific fragments or variants can be produced in host cells, transgenic animals or transgenic plants, and administered to the individual in crude, partially purified, or substantially pure form.

In another embodiment, the invention embraces methods of treating Friedreich's ataxia or Leigh's syndrome, comprising administering, to an individual having a mitochondrial disorder that is not a respiratory chain disorder, a therapeutically effective amount of a composition comprising one or more EPO mimetic hinge core mimetibody polypeptides or specified fragments or variants thereof, including isolated nucleic acids that encode at least one EPO mimetic hinge core mimetibody or specific fragments or variants, vectors, host cells, transgenic animal or plants

In some embodiments, the EPO mimetic molecule is an EPO mimetic antibody fusion protein as described in U.S. Pat. No. 7,241,733 or US 2006/0051844, incorporated herein by reference in their entirety.

In other embodiments, the molecule having EPO activity is an EPO-mimetic antibody fusion protein such as CNTO-528 or CNTO-530.

In other embodiments, the one or more molecules administered to the individual with a mitochondrial disease that is not a respiratory chain disease are molecules that are capable of increasing the endogenous EPO or stimulating erythropoiesis.

In other embodiments, the invention embraces methods of treating a mitochondrial disorder, comprising administering, to an individual having a mitochondrial disorder that is not a respiratory chain disorder, a therapeutically effective amount of a composition comprising a molecule capable of increasing the endogenous EPO or stimulating erythropoiesis, wherein said molecule stabilizes the alpha subunit of hypoxia inducible factor (HIF-α).

In other embodiments, the invention embraces methods of treating a mitochondrial disorder, comprising administering, to an individual having a mitochondrial disorder that is not a respiratory chain disorder, a therapeutically effective amount of a composition comprising a molecule capable of increasing the endogenous EPO or stimulating erythropoiesis, wherein said molecule inhibits hydroxylation of HIF-α or inhibits HIF prolyl hydroxylase enzyme activity.

In other embodiments, the invention embraces methods of treating a mitochondrial disorder, comprising administering, to an individual having a mitochondrial disorder that is not a respiratory chain disorder, a therapeutically effective amount of a composition comprising a molecule capable of increasing the endogenous EPO or stimulating erythropoiesis, wherein said molecule inhibits 2-oxoglutarate dioxygenase enzyme activity.

In other embodiments, the invention embraces methods of treating a mitochondrial disorder that is not a respiratory chain disorder, comprising administering, to an individual having a mitochondrial disorder that is not a respiratory chain disorder, a therapeutically effective amount of a composition comprising a molecule capable of increasing the endogenous EPO or stimulating erythropoiesis, wherein said molecule inhibits hydroxylation of HIF-α or inhibits HIF prolyl hydroxylase enzyme activity.

In particular embodiments, the molecules capable of increasing the endogenous EPO or stimulating erythropoiesis are selected from FG-2216, FG-4539, FG-4592 and FG-6513.

In other embodiments, the invention embraces methods of treating a mitochondrial disorder, comprising administering, to an individual having a mitochondrial disorder that is not a respiratory chain disorder, a therapeutically effective amount of a composition comprising the erythropoiesis stimulating agent Hematide™, (Hematide is a registered trademark of Affymax, Inc., Palo Alto, Calif., USA, for a pharmaceutical preparation for use in stimulating human blood cell production) a synthetic, pegylated peptidic compound that binds to and activates the erythropoietin receptor.

In another embodiment, the invention embraces methods of treating a mitochondrial disorder that is not a respiratory chain disorder, comprising administering a therapeutically effective amount of a composition comprising one or more small molecule EPO mimetic molecules to an individual with a mitochondrial dysfunction implicated in Parkinson's disease, Alzheimer's disease, amyotrophic lateral sclerosis (ALS) or Huntington's disease.

In another embodiment, the individual with a mitochondrial dysfunction has cancer.

In another embodiment, the individual with a mitochondrial dysfunction is a child suffering from autism, mental retardation, developmental delay, failure to thrive and growth failure.

In another embodiment the individual with a mitochondrial dysfunction has cardiac dysfunction manifestations including dilated or hypertrophic cardiomyopathy, cardiac arrhythmias and conduction defects.

In another embodiment, the individual with a mitochondrial dysfunction has muscle dysfunction including fatigue, exercise intolerance and weakness, myalgias, rhabdomyolyis, and hypotomia.

In another embodiment, the individual with a mitochondrial dysfunction has hepatopathic manifestations including neonatal liver failure, hepatic steatohepatitis, cholestasis and chronic liver failure.

In other embodiments, the individual with a mitochondrial dysfunction has an endocrine disorder, such as diabetes mellitus or other endocrine disorders.

In another aspect, the invention embraces methods of treating Friedreich's ataxia, comprising administering a therapeutically effective amount of a composition comprising one or more EPO mimetic molecules, to an individual in need of such treatment. In some embodiments the individual is administered a therapeutically effective amount of a composition comprising CNTO-528 or CNTO-530.

In another aspect, the invention embraces methods of treating Friedreich's ataxia, comprising administering a therapeutically effective amount of a composition comprising one or more molecules capable of increasing the endogenous EPO or stimulating erythropoiesis, to an individual in need of such treatment. In some embodiments the individual is administered a therapeutically effective amount of a composition comprising a molecule capable of increasing the endogenous EPO or stimulating erythropoiesis, wherein said molecule is selected from FG-2216, FG-4539, FG-4592 and FG-6513. In another embodiment said molecule is Hematide™.

In another aspect, the invention embraces methods of treating Leigh's syndrome, comprising administering a therapeutically effective amount of a composition comprising one or more EPO mimetic molecules to an individual with Leigh's syndrome. In some embodiments the individual is administered a therapeutically effective amount of a composition comprising CNTO-528 or CNTO-530.

In another aspect, the invention embraces methods of treating Leigh's syndrome, comprising administering a therapeutically effective amount of a composition comprising one or more molecules capable of increasing the endogenous EPO or stimulating erythropoiesis, to an individual in need of such treatment. In some embodiments the individual is administered a therapeutically effective amount of a composition comprising a molecule capable of increasing the endogenous EPO or stimulating erythropoiesis, wherein said molecule is selected from FG-2216, FG-4539, FG-4592 and FG-6513. In another embodiment said molecule is Hematide™.

In another aspect, the invention embraces a method of treating a neurodegenerative disease caused by acquired mitochondrial dysfunction, comprising administering a therapeutically effective amount of a composition comprising one or more EPO mimetic molecules or molecules capable of increasing the endogenous EPO to an individual with a neurodegenerative disease caused by acquired mitochondrial dysfunction. In some embodiments the composition comprises one or more EPO mimetic molecules, such as CNTO-528 or CNTO-530. In other embodiments, the composition comprises one or more molecules capable of increasing the endogenous EPO, such as inhibitors of hypoxia inducible factor prolyl hydroxylase, such as FG-2216, FG-4539, FG-4592 and FG-6513. In another embodiment, the composition comprises Hematide™.

In any of the foregoing methods, the therapeutically effective amount can be an amount sufficient to improve one or more energy biomarker levels, such as pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species, or exercise tolerance, to within about at least two standard deviations of normal in a subject, more preferably within about at least one standard deviation of normal in a subject, within about at least one-half standard deviation of normal, or within about at least one-quarter standard deviation of normal. When an increase in energy biomarker levels is desired for improvement, the levels or one or more energy biomarkers are increased as indicated above; when a decrease in energy biomarker levels is desired for improvement, the levels of one or more energy biomarkers are decreased as indicated above. In another embodiment of any of the foregoing methods, when an increase in the levels of one or more energy biomarkers is desirable, the therapeutically effective amount can be an amount sufficient to increase the levels of the one or more energy biomarker by about at least 10% above the subject's level of the respective one or more energy biomarkers before treatment, by about at least 20% above the subject's level of the respective one or more energy biomarkers before treatment, by about at least 30% above the subject's level of the respective one or more energy biomarkers before treatment, by about at least 40% above the subject's level of the respective one or more energy biomarkers before treatment, by about at least 50% above the subject's level of the respective one or more energy biomarkers before treatment, by about at least 75% above the subject's level of the respective one or more energy biomarkers before treatment, or by about at least 100% above the subject's level of the respective one or more energy biomarkers before treatment. In another embodiment of any of the foregoing methods, when a decrease in a level of one or more energy biomarkers is desired, the level of the one or more energy biomarkers can be decreased by about at least 10% below the subject's level of the respective one or more energy biomarkers before treatment, by about at least 20% below the subject's level of the respective one or more energy biomarkers before treatment, by about at least 30% below the subject's level of the respective one or more energy biomarkers before treatment, by about at least 40% below the subject's level of the respective one or more energy biomarkers before treatment, by about at least 50% below the subject's level of the respective one or more energy biomarkers before treatment, by about at least 75% below the subject's level of the respective one or more energy biomarkers before treatment, or by about at least 90% below the subject's level of the respective one or more energy biomarkers before treatment.

### MODES FOR CARRYING OUT THE INVENTION

By “respiratory chain” is meant the components (including, but not limited to, proteins, tetrapyrroles, and cytochromes) comprising mitochondrial complex I, II, III, IV, and/or V; “respiratory chain protein” refers to the protein components of those complexes.

By “therapeutically effective amount” is meant an amount sufficient to provide a measurable increase in the utilization of oxygen in an individual; and/or an amount sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

Erythropoietin (EPO) has been the focus of significant research activity due to its utility in treating several serious diseases. EPO is currently approved in the United States for treatment of anemia in patients with chronic renal failure undergoing dialysis (recombinant human erythropoietin is sold under the brand name Epogen®, a registered trademark of Amgen, Inc., Thousand Oaks, Calif.). EPO is also believed to be useful in treatment of various other disorders; see, e.g., International Patent Application No. WO 2006/006165, directed to using EPO for enhancing immune responses and for the treatment of certain lympho-proliferative disorders; US 2006/0094648, directed to therapeutic or prophylactic treatment of myocardial ischemia, such as due to myocardial infarction, by administering erythropoietin; or US 2005/0272634, directed to using EPO for treatment of various disorders such as hypercholesterolemia, atherosclerosis, and diabetes. EPO or molecules having sequence homology to EPO are not encompassed in this invention. In one embodiment, the molecules used in this invention have less than about 40% sequence homology to EPO. In another embodiment, the molecules used in this invention have less than about 30% sequence homology to EPO. In another embodiment, the molecules used in this invention have less than about 20% sequence homology to EPO. In another embodiment, the molecules used in this invention have less than about 10% sequence homology to EPO. In another embodiment, the molecules used in this invention have less than about 30% sequence identity to EPO. In another embodiment, the molecules used in this invention have less than about 20% sequence identity to EPO. In another embodiment, the molecules used in this invention have less than about 10% sequence identity to EPO. In any of the foregoing embodiments, sequence homology or sequence identity between two sequences can be measured using the BLAST algorithm (Altschul, S. F., Gish, W., Miller, W., Myers, E. W. & Lipman, D. J. (1990) “Basic local alignment search tool.” J. Mol. Biol. 215:403-410, available at World Wide web address blast.ncbi.nlm.nih.gov/Blast.cgi?PAGE_TYPE=BlastSearch&PROG_DEF=blastn&BLA ST_PROG_DEF=megaBlast&BLAST_SPEC=blast2seq, where the homology or identity is computed relative to the entire sequence of EPO. The BLOSUM or PAM matrices can be used for scoring, such as BLOSUM62, BLOSUM80, BLOSUM45, or PAM70, with word size set to 3 or 2, and expectation value of 10, gap existence penalties of between 7 and 12 (e.g., 11), and gap extension penalties of 1 or 2.

In the spirit of this invention, molecules having erythropoietin (EPO) activity refer to polypeptides and proteins, and small molecules having at least one of the biological activities of human erythropoietin, but that do not have the amino acid sequence of erythropoietin, are not homologous to erythropoietin, or are not derivatives with sugar residues or any mutant or variant of erythropoietin. For example, the molecules described in US 2004/0157293 or in Leist et al., Science Vol 305, 239-242 which have homology to EPO are excluded. Molecules having erythropoietin activity include, but are not limited to, erythropoietin mimetics, erythropoietin fragments, hybrid erythropoietin proteins, mutants and variants of any of the foregoing molecules, where the molecules are not similar to EPO itself (i.e., are not homologous to EPO) regardless of the biological activity of the same and further regardless of the method of synthesis or manufacture thereof. By a composition (or molecule, etc.) having “erythropoietin activity” is meant any composition (or molecule, etc.) having the full range of biological activity of human erythropoietin or at least one of the biological activities of EPO, such as the in vivo or in vitro activity of causing an increase in production of reticulocytes and/or red blood cells by bone marrow cells.

“Erythropoietin-mimetics” or “EPO-mimetics” are molecules capable of acting as EPO in binding to the EPO receptor (EPO-R) wherein the mimetic has no similarity to native EPO. EPO mimetics are well known to those skilled in the art. Two kinds of EPO-mimetics have been described: peptides and non-peptides. Specific examples of erythropoietin mimetics are described in U.S. Pat. No. 5,767,078 and U.S. Pat. No. 5,773,569. Additional EPO-mimetics, such as CNTO-528, and CNTO-530 have been produced using Centocor's technology Mimetibody™ and described, for example, in PCT publications WO 08/042800 and WO 07/115148, U.S. Pat. No. 7,241,733 and US patent publication US 2006/0051844. CNTO-530 is a 58 kD antibody Fc domain fusion protein, that contains two EMP1 sequences as a pharmacophore. CNTO-530 has no sequence homology with EPO but acts as a novel erythropoietin receptor agonist.

Small molecule EPO mimetics were discovered by scientists from Scripps, Affymax, and Johnson Pharmaceutical Research Institute screening a peptide phage library to search for novel sequences that bound to EPO-R. One product resulting from this research is a pegylated peptide with no sequence homology to EPO but with EPO-R specificity, marketed as Hematide™. Some of these agents are described in Bunn, Blood, (2007) Vol 109 No. 3, 808-873.

By “molecule capable of increasing the endogenous EPO or stimulating erythropoiesis” is meant molecules that regulate the EPO gene and/or the interaction of EPO with EPO-R, and which excludes EPO or molecules having sequence homology to EPO. These molecules can be proteins or peptides, or small molecules. Rather than being agents that directly stimulate and produce erythropoiesis by combining with the erythropoietin receptor, they actually cause the production of endogenous erythropoietin. By producing the erythropoietin, the agents are able to sustain lower but more sustained concentration of EPO, and it is the endogenous erythropoietin which then produces the erythropoiesis.

“Erythropoiesis stimulating agents” (ESA) are substances that upregulate genes for, and/or expression and/or activity of, proteins besides EPO that are important in erythropoiesis including EPO-R, transferrin, transferrin receptor, or ferroportin. Only erythropoiesis stimulating agents with no EPO sequence homology are included in this invention. Some of these agents are also molecules which can be orally administered. The most advanced development of an oral ESA is a group of compounds originating from Fibrogen, now in co-development with Astellas for certain territories, now including Europe. These compounds up-regulate endogenous EPO by inhibition of hypoxia induced factor prolyl hydroxylase-(HIF-PH). They include FG-2216, FG-4539, FG-4592 and FG-6513. Some of these compounds are disclosed in US Publications US 2006/0178317, US 2006/0178316 and US 2006/0183695 and PCT publication WO 03/049686, WO 05/011696, WO 06/133391 and WO 07/146438, incorporated herein in their entirety.

By “variant” is meant a modified peptide that retains its binding properties wherein the modifications include, but are not limited to, conservative substitutions in which one or more amino acids are substituted for other amino acids; deletion or addition of amino acids that have minimal influence on the binding properties or secondary structure, conjugation of a linker; and post-translation modifications such as, for example, the addition of functional groups. Conservative amino acid substitution is an amino acid substituted by an alternative amino acid of similar charge density, hydrophilicity/hydrophobicity, size, and/or configuration (e.g. Val for Ile). Means of making such modifications are well known in the art.

EPO-mimetics or specified portions or variants thereof, molecules with EPO activity, and molecules capable of increasing endogenous EPO or stimulating erythropoiesis (referred to herein as “compounds for use in the invention”) can be administered to a subject via parenteral administration, including, but not limited to, intravenous, intramuscular, subcutaneous, intraperitoneal, intracerebral, intraventricular, intracerebroventricular, intrathecal, intracisternal, intraspinal and perispinal administration. Compounds for use in the invention can also be delivered continuously or semi-continuously via pump devices. Compounds for use in the invention can also be delivered as “long-acting compounds” including sustained-release compositions and formulations with increased circulating half-life, typically achieved through modification such as reducing immunogenicity and clearance rate, and encapsulation in polymer microspheres. The route of administration can be selected by the health care professional in accordance with known principles. When a compound for use in the invention is administered, the formulation, dosage, and route of administration are also determined by the health care professional in accordance with known principles; the energy biomarkers described herein can be used to monitor efficacy of treatment.

Any method of the present invention can comprise a method for treating a mitochondrial disorder, comprising administering an effective amount of a composition or pharmaceutical composition comprising at least compound for use in the invention to a cell, tissue, organ, animal or patient in need of such modulation, treatment or therapy.

Typically, treatment of mitochondrial conditions is effected by administering an effective amount or dosage of at least one compound for use in the invention that totals, on average, a range from at least about 0.01 to 500 milligrams of at least one compound for use in the invention/kilogram of patient per dose, and preferably from at least about 0.1 to 100 milligrams compound for use in the invention/kilogram of patient per single or multiple administrations, depending upon the specific activity of compound(s) contained in the composition. Alternatively, the effective serum concentration can comprise about 0.1-5000 μg/ml serum concentration per single or multiple administrations. Suitable dosages are known to medical practitioners and will depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include about 0.01, 0.02, 0.03, 0.04, 0.05, 0.06, 0.07, 0.08, 009, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, and/or 30 mg/g/administration, or any range, value or fraction thereof, or to achieve a serum concentration of about 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 mg/kg of body weight. Ordinarily about 0.1 to 50 mg/kg, and preferably about 0.1 to 10 mg/kg per administration or in sustained release form is effective to obtain desired results.

As a non-limiting example, treatment of humans or animals can be provided as a one-time or periodic dosage of at least one compound for use in the invention of about 0.01 to 100 mg/kg, such as about 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, or at least one administration per day of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/kg, or alternatively, at least administration per week of about 1, 2, 3, 4, 5, 6, 7, 8, 9, 10, 11, 12, 13, 14, 15, 16, 17, 18, 19 or 20 mg/kg, or any combination thereof, using single, infusion or repeated doses.

Dosage forms (composition) suitable for internal administration generally contain from about 0.0001 milligram to about 500 milligrams of active ingredient per unit or container. In these pharmaceutical compositions the active ingredient will ordinarily be present in an amount of about 0.5-95% by weight based on the total weight of the composition.

For parenteral administration, the compound for use in the invention can be formulated as a solution, suspension, emulsion or lyophilized powder in association, or separately provided, with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Therapeutic Administration

Many known and developed modes of administration can be used according to the present invention for administering pharmaceutically effective amounts of at least one compound for use in the invention according to the present invention. While pulmonary administration is used in the following description, other modes of administration can be used according to the present invention with suitable results.

A compound for use in the invention can be delivered in a carrier, as a solution, emulsion, colloid, or suspension, or as a powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semi-synthetic fatty oils or fatty acids; natural or synthetic or semi-synthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of at least one compound for use in the invention by parenteral, subcutaneous, intramuscular, intravenous, bolus, vaginal, rectal, buccal, sublingual, intranasal, or transdermal means. Compositions containing compounds for use in the invention can be prepared for use for parenteral (subcutaneous, intramuscular or intravenous) administration particularly in the form of liquid solutions or suspensions; for use in vaginal or rectal administration, particularly in semisolid forms such as creams and suppositories; for buccal or sublingual administration particularly in the form of tablets or capsules; or intranasal administration particularly in the form of powders, nasal drops or aerosols or certain agents; or transdermal administration particularly in the form of a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

### Pulmonary/Nasal Administration

For pulmonary administration, preferably at least one compound for use in the invention is delivered in a particle size effective for reaching the lower airways of the lung or sinuses. According to the invention, at least one compound for use in the invention can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of compounds for use in the invention are also known in the art. All such devices can use of formulations suitable for the administration for the dispensing of compounds for use in the invention in an aerosol. Such aerosols can be comprised of either solutions (both aqueous and non aqueous) or solid particles. Metered dose inhalers like the Ventolin™ (Glaxo Group Ltd) metered dose inhaler, typically use a propellant gas and require actuation during inspiration (See, e.g., WO 94/16970, WO 98/35888). Dry powder inhalers like Turbuhaler™ (Astra), Monohaler™ (Miat SpA), Rotahaler™ (Glaxo), Diskus™ (Glaxo), Spiros™ inhaler (Dura), devices marketed by Inhale Therapeutics, and the Spinhaler™ powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent™ nebulizer (Mallinckrodt), and the Acorn II™ nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one compound for use in the invention is delivered by a dry powder inhaler or a sprayer. There are several desirable features of an inhalation device for administering at least one EPO mimetic or specified portion or variant of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of EPO mimetic or specified portion or variant Compositions as a Spray

A spray including a compound for use in the invention can be produced by forcing a suspension or solution of at least one compound for use in the invention through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one compound for use in the invention delivered by a sprayer have a particle size less than about 10 μm, preferably in the range of about 1 μm to about 5 μm, and most preferably about 2 μm to about 3 μm.

Formulations of at least one compound for use in the invention suitable for use with a sprayer typically include a compound for use in the invention in an aqueous solution at a concentration of about 1 mg to about 20 mg of at least one compound for use in the invention per ml of solution. The formulation can include agents such as an excipient, a buffer, an isotonicity agent, a preservative, a surfactant, and, preferably, zinc. The formulation can also include an excipient or agent for stabilization of the compound for use in the invention, such as a buffer, a reducing agent, a bulk protein, or a carbohydrate. Bulk proteins useful in formulating compounds for use in the invention include albumin, protamine, or the like. Typical carbohydrates useful in formulating compounds for use in the invention include sucrose, mannitol, lactose, trehalose, glucose, or the like. The formulation of a compound for use in the invention can also include a surfactant, which can reduce or prevent surface-induced aggregation caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation compounds for use in the invention can also be included in the formulation.

### Administration of Compounds for Use in the Invention by a Nebulizer

Compounds for use in the invention can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of a compound for use in the invention through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of one or more compounds for use in the invention either directly or through a coupling fluid, creating an aerosol including the one or more compounds for use in the invention. Advantageously, particles of the one or more compounds for use in the invention delivered by a nebulizer have a particle size less than about 10 μm, preferably in the range of about 1 μm to about 5 μm, and most preferably about 2 μm to about 3 μm.

Formulations of at least one compound for use in the invention suitable for use with a nebulizer, either jet or ultrasonic, typically include at least compound for use in the invention in an aqueous solution at a concentration of about 1 mg to about 20 mg of at least one compound for use in the invention per ml of solution. The formulation can include agents such as an excipient, a buffer, an isotonicity agent, a preservative, a surfactant, and, preferably, zinc. The formulation can also include an excipient or agent for stabilization of the at least one compound for use in the invention, such as a buffer, a reducing agent, a bulk protein, or a carbohydrate. Bulk proteins useful in formulating at least one compound for use in the invention include albumin, protamine, or the like. Typical carbohydrates useful in formulating at least one compound for use in the invention include sucrose, mannitol, lactose, trehalose, glucose, or the like. The at least one compound for use in the invention can also include a surfactant, which can reduce or prevent surface-induced aggregation of the at least one compound for use in the invention caused by atomization of the solution in forming an aerosol. Various conventional surfactants can be employed, such as polyoxyethylene fatty acid esters and alcohols, and polyoxyethylene sorbitan fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. When one or more of the compounds for use in the invention is a protein, additional agents known in the art for formulation of proteins can also be included in the formulation.

### Administration of Compositions Comprising Compounds for Use in the Invention by a Metered Dose Inhaler

In a metered dose inhaler (MDI) a propellant, at least one compound for use in the invention, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of compounds for use in the invention produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of compounds for use in the invention for use with a metered-dose inhaler device will generally include a finely divided powder containing at least one compound for use in the invention as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one compound for use in the invention as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. When the one or more compounds for use in the invention is a protein, additional agents known in the art for formulation of a protein can also be included in the formulation.

One of ordinary skill in the art will recognize that the methods of the current invention can be achieved by pulmonary administration of a composition comprising at least one compound for use in the invention via devices not described herein.

### Mucosal Formulations and Administration

For absorption through mucosal surfaces, compositions and methods of administering at least one compound for use in the invention include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like.

### Oral Formulations and Administration

Formulations for oral administration rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 are used to deliver biologically active agents orally are known in the art.

### Transdermal Formulations and Administration

For transdermal administration, the at least one compound for use in the invention is encapsulated in a delivery device such as a liposome or polymeric nanoparticles, micro particle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

### Prolonged Administration and Formulations

It can be sometimes desirable to deliver the compounds for use in the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds for use in the invention that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds for use in the invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. No. 5,770,222 and “Sustained and Controlled Release Drug Delivery Systems”, J. R. Robinson ed., Marcel Dekker, Inc., N.Y., 1978).

### Clinical Assessment of Mitochondrial Diseases and Efficacy of Therapy

Several readily measurable clinical markers are used to assess the metabolic state of patients with mitochondrial disorders. These markers can also be used as indicators of the efficacy of a given therapy, as the level of a marker is moved from the pathological value to the healthy value. These clinical markers include, but are not limited to, one or more energy biomarkers such as lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; anaerobic threshold; reduced coenzyme Q (CoQ^{red}) levels; oxidized coenzyme Q (CoQ^{ox}) levels; total coenzyme Q (CoQ^{tot}) levels; oxidized cytochrome c levels; reduced cytochrome c levels; oxidized cytochrome c/reduced cytochrome c ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; and levels of oxygen consumption (VO2), levels of carbon dioxide output (VCO2), and respiratory quotient (VCO2/VO2). Several of these clinical markers are measured routinely in exercise physiology laboratories, and provide convenient assessments of the metabolic state of a subject. In one embodiment of the invention, the level of one or more energy biomarkers in a patient suffering from a mitochondrial disease, such as FRDA, is improved to within two standard deviations of the average level in a healthy subject. In another embodiment of the invention, the level of one or more of these energy biomarkers in a patient suffering from a mitochondrial disease, such as FRDA is improved to within one standard deviation of the average level in a healthy subject. Exercise intolerance can also be used as an indicator of the efficacy of a given therapy, where an improvement in exercise tolerance (i.e., a decrease in exercise intolerance) indicates efficacy of a given therapy.

Several metabolic biomarkers have already been used to evaluate efficacy of CoQ10, and these metabolic biomarkers can be monitored as energy biomarkers for use in the methods of the current invention. Pyruvate, a product of the anaerobic metabolism of glucose, is removed by reduction to lactic acid in an anaerobic setting or by oxidative metabolism, which is dependent on functional mitochondria. Dysfunction of the mitochondria may lead to inadequate removal of lactate and pyruvate from the circulation and elevated lactate/pyruvate ratios are observed in mitochondrial cytopathies (see Scriver, The Metabolic and Molecular Bases of Inherited Disease, 7th ed., New York: McGraw-Hill, Health Professions Division, 1995; and Munnich et al., J. Inherit. Metab. Dis. 15(4):448-55 (1992)). Blood lactate/pyruvate ratio (Chariot et al., Arch. Pathol. Lab. Med. 118(7):695-7 (1994)) is, therefore, widely used as a noninvasive test for detection of mitochondrial cytopathies (see Scriver, The Metabolic and Molecular Bases of Inherited Disease, 7th ed., New York: McGraw-Hill, Health Professions Division, 1995; and Munnich et al., J. Inherit. Metab. Dis. 15(4):448-55 (1992)) and toxic mitochondrial myopathies (Chariot et al., Arthritis Rheum. 37(4):583-6 (1994)). Changes in the redox state of liver mitochondria can be investigated by measuring the arterial ketone body ratio (acetoacetate/3-hydroxybutyrate: AKBR) (Ueda et al., J. Cardiol. 29(2):95-102 (1997)). Urinary excretion of 8-hydroxy-2′-deoxyguanosine (8-OHdG) often has been used as a biomarker to assess the extent of repair of ROS-induced DNA damage in both clinical and occupational settings (Erhola et al., FEBS Lett. 409(2):287-91 (1997); Honda et al., Leuk. Res. 24(6):461-8 (2000); Pilger et al., Free Radic. Res. 35(3):273-80 (2001); Kim et al. Environ Health Perspect 112(6):666-71 (2004)).

Magnetic resonance spectroscopy (MRS) has been useful in the diagnoses of mitochondrial cytopathy by demonstrating elevations in cerebrospinal fluid (CSF) and cortical white matter lactate using proton MRS (1H-MRS) (Kaufmann et al., Neurology 62(8):1297-302 (2004)). Phosphorous MRS (31P-MRS) has been used to demonstrate low levels of cortical phosphocreatine (PCr) (Matthews et al., Ann. Neurol. 29(4):435-8 (1991)), and a delay in PCr recovery kinetics following exercise in skeletal muscle (Matthews et al., Ann. Neurol. 29(4):435-8 (1991); Barbiroli et al., J. Neurol. 242(7):472-7 (1995); Fabrizi et al., J. Neurol. Sci. 137(1):20-7 (1996)). A low skeletal muscle PCr has also been confirmed in patients with mitochondrial cytopathy by direct biochemical measurements.

Exercise testing is particularly helpful as an evaluation and screening tool in mitochondrial myopathies. One of the hallmark characteristics of mitochondrial myopathies is a reduction in maximal whole body oxygen consumption (VO2max) (Taivassalo et al., Brain 126(Pt 2):413-23 (2003)). Given that VO2max is determined by cardiac output (Qc) and peripheral oxygen extraction (arterial-venous total oxygen content) difference, some mitochondrial cytopathies affect cardiac function where delivery can be altered; however, most mitochondrial myopathies show a characteristic deficit in peripheral oxygen extraction (A-VO2 difference) and an enhanced oxygen delivery (hyperkinetic circulation) (Taivassalo et al., Brain 126(Pt 2):413-23 (2003)). This can be demonstrated by a lack of exercise induced deoxygenation of venous blood with direct AV balance measurements (Taivassalo et al., Ann. Neurol. 51(1):38-44 (2002)) and non-invasively by near infrared spectroscopy (Lynch et al., Muscle Nerve 25(5):664-73 (2002); van Beekvelt et al., Ann. Neurol. 46(4):667-70 (1999)).

Several of these energy biomarkers are discussed in more detail as follows. It should be emphasized that, while certain energy biomarkers are discussed and enumerated herein, the invention is not limited to modulation, normalization or enhancement of only these enumerated energy biomarkers.

Lactic acid (lactate) levels: Mitochondrial dysfunction typically results in abnormal levels of lactic acid, as pyruvate levels increase and pyruvate is converted to lactate to maintain capacity for glycolysis. Mitochondrial dysfunction can also result in abnormal levels of NADH+H⁺, NADPH+H⁺, NAD, or NADP, as the reduced nicotinamide adenine dinucleotides are not efficiently processed by the respiratory chain. Lactate levels can be measured by taking samples of appropriate bodily fluids such as whole blood, plasma, or cerebrospinal fluid. Using magnetic resonance, lactate levels can be measured in virtually any volume of the body desired, such as the brain.

Measurement of cerebral lactic acidosis using magnetic resonance in MELAS patients is described in Kaufmann et al., Neurology 62(8):1297 (2004). Values of the levels of lactic acid in the lateral ventricles of the brain are presented for two mutations resulting in MELAS, A3243G and A8344G. Whole blood, plasma, and cerebrospinal fluid lactate levels can be measured by commercially available equipment such as the YSI 2300 STAT Plus Glucose & Lactate Analyzer (YSI Life Sciences, Ohio).

NAD, NADP, NADH and NADPH levels: Measurement of NAD, NADP, NADH (NADH+H⁺) or NADPH (NADPH+H⁺) can be measured by a variety of fluorescent, enzymatic, or electrochemical techniques, e.g., the electrochemical assay described in US 2005/0067303.

Oxygen consumption (vO₂ or VO2), carbon dioxide output (vCO₂ or VCO2), and respiratory quotient (VCO2/VO2): VO2 is usually measured either while resting (resting vO₂) or at maximal exercise intensity (vO₂ max). Optimally, both values will be measured. However, for severely disabled patients, measurement of vO₂ max may be impractical. Measurement of both forms of vO₂ is readily accomplished using standard equipment from a variety of vendors, e.g., Korr Medical Technologies, Inc. (Salt Lake City, Utah). VCO2 can also be readily measured, and the ratio of VCO2 to VO2 under the same conditions (VCO2/VO2, either resting or at maximal exercise intensity) provides the respiratory quotient (RQ).

Oxidized Cytochrome c, reduced Cytochrome c, and ratio of oxidized Cytochrome c to reduced Cytochrome c: Cytochrome c parameters, such as oxidized cytochrome c levels (Cyt Coₒₓ), reduced cytochrome c levels (Cyt C_{red}), and the ratio of oxidized cytochrome c/reduced cytochrome c ratio (Cyt Cₒₓ)/(Cyt C_{red}), can be measured by in vivo near infrared spectroscopy. See, e.g., Rolfe, P., “In vivo near-infrared spectroscopy,” Ann. Rev. Biomed. Eng. 2:715-54 (2000) and Strangman et al., “Non-invasive neuroimaging using near-infrared light” Biol. Psychiatry 52:679-93 (2002).

Exercise tolerance/Exercise intolerance: Exercise intolerance is defined as “the reduced ability to perform activities that involve dynamic movement of large skeletal muscles because of symptoms of dyspnea or fatigue” (Piña et al., Circulation 107:1210 (2003)). Exercise intolerance is often accompanied by myoglobinuria, due to breakdown of muscle tissue and subsequent excretion of muscle myoglobin in the urine. Various measures of exercise intolerance can be used, such as time spent walking or running on a treadmill before exhaustion, time spent on an exercise bicycle (stationary bicycle) before exhaustion, and the like. Treatment with the methods of the invention can result in about a 10% or greater improvement in exercise tolerance (for example, about a 10% or greater increase in time to exhaustion, e.g., from 10 minutes to 11 minutes), about a 20% or greater improvement in exercise tolerance, about a 30% or greater improvement in exercise tolerance, about a 40% or greater improvement in exercise tolerance, about a 50% or greater improvement in exercise tolerance, about a 75% or greater improvement in exercise tolerance, or about a 100% or greater improvement in exercise tolerance. While exercise tolerance is not, strictly speaking, an energy biomarker, for the purposes of the invention, it can be used to evaluate therapeutic efficacy.

Similarly, tests for normal and abnormal values of pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species are known in the art and can be used to evaluate efficacy of therapeutic intervention.

Partial or complete suppression of the mitochondrial disease can result in a lessening of the severity of one or more of the symptoms that the subject would otherwise experience. For example, partial suppression of FRDA could result in the reduction or halt of progressive loss of voluntary motor coordination. Similarly, partial suppression of Leigh's syndrome could result in the reduction in the number of seizure episodes suffered.

Any one or any combination of the energy biomarkers described herein provides conveniently measurable benchmarks by which to gauge the effectiveness of treatment or suppressive therapy. Additionally, other energy biomarkers are known to those skilled in the art and can be monitored to evaluate the efficacy of treatment or suppressive therapy. Again, while exercise tolerance is not, strictly speaking, an energy biomarker, for the purposes of the invention, it can be used to evaluate therapeutic efficacy, such as for the discussion below regarding increases or decreases in energy biomarkers.

When an increase in the level of one or more of the energy biomarkers is desired, the level of the energy biomarker can be increased to within about at least two standard deviations of normal in a subject, more preferably increased to within about at least one standard deviation of normal in a subject, increased to within about at least one-half standard deviation of normal, or increased to within about at least one-quarter standard deviation of normal, by treatment with a composition having EPO activity according to the invention. Alternatively, the level can be increased by about at least 10% above the subject's level of the respective one or more energy biomarkers before treatment, by about at least 20% above the subject's level of the respective one or more energy biomarkers before treatment, by about at least 30% above the subject's level of the respective one or more energy biomarkers before treatment, by about at least 40% above the subject's level of the respective one or more energy biomarkers before treatment, by about at least 50% above the subject's level of the respective one or more energy biomarkers before treatment, by about at least 75% above the subject's level of the respective one or more energy biomarkers before treatment, or by about at least 100% above the subject's level of the respective one or more energy biomarkers before treatment.

When a decrease in a level of one or more energy biomarkers is desired, the level of the one or more energy biomarkers can be decreased to a level within about at least two standard deviations of normal in a subject, more preferably decreased to within about at least one standard deviation of normal in a subject, decreased to within about at least one-half standard deviation of normal, or decreased to within about at least one-quarter standard deviation of normal, by treatment with a composition having EPO activity according to the invention. Alternatively, the level of the one or more energy biomarkers can be decreased by about at least 10% below the subject's level of the respective one or more energy biomarkers before treatment, by about at least 20% below the subject's level of the respective one or more energy biomarkers before treatment, by about at least 30% below the subject's level of the respective one or more energy biomarkers before treatment, by about at least 40% below the subject's level of the respective one or more energy biomarkers before treatment, by about at least 50% below the subject's level of the respective one or more energy biomarkers before treatment, by about at least 75% below the subject's level of the respective one or more energy biomarkers before treatment, or by about at least 90% below the subject's level of the respective one or more energy biomarkers before treatment.

### BIOLOGICAL EXAMPLES

### Example A

Fibroblasts from Friedreich's Ataxia Patients.

Primary human fibroblasts obtained from patients with Friedreich's Ataxia (FRDA) purchased from the Coriell Cell Repositories (Camden, N.J.; repository number GM04078) were grown in 10 cm tissue culture plates. Every third day, they were split at a 1:3 ratio. Human dermal fibroblasts from mitochondrial disease patients have been shown to be hypersensitive to inhibition of the de novo synthesis of glutathione (GSH) with L-buthionine-(S,R)-sulfoximine (BSO), a specific inhibitor of GSH synthetase (Jauslin et al., Hum. Mol. Genet. (2002)11(24):3055). FRDA fibroblasts were stressed by addition of L-buthionine-(S,R)-sulfoximine (BSO), as described in Jauslin et al., Hum. Mol. Genet. (2002)11(24):3055, Jauslin et al., FASEB J. (2003)17:1972-4, and International Patent Application WO 2004/003565, such that cellular viability of FRDA but not of healthy patient fibroblasts, was decreased. Prior to stress, cells were pre-treated with an EPO mimetic compound and cellular viability was monitored. Increased cellular viability suggested that EPO-mimetic affects cellular susceptibility to oxidative stress by modulating overall cellular health.

### Materials:

MEM Medium 199 with Earle's Balanced Salts and Fetal Calf Serum (Invitrogen, Carlsbad Calif.)

Basic fibroblast growth factor and epidermal growth factor (PeproTech, Rocky Hill, N.J.).

Penicillin-streptomycin-glutamine mix (Sigma, St Louis, Mo.),

L-buthionine (S,R)-sulfoximine (Sigma, St Louis, Mo.)

Insulin from bovine pancreas (Sigma, St Louis, Mo.)

Calcein AM (Anaspec, San Jose, Calif).

### Procedure:

Cell culture medium was made by combining 125 ml M199, 50 ml Fetal Calf Serum, 100 U/ml penicillin, 100 ug/ml streptomycin, 2 mM glutamine, 10 ug/ml insulin, 10 ng/ml EGF, and 10 ng/ml bFGF; MEM was added to make the volume up to 500 ml. A 10 mM BSO solution was prepared by dissolving 444 mg BSO in 200 ml of medium with subsequent filter-sterilization.

During the course of the experiments, this solution was stored at +4° C.

A culture with FRDA fibroblasts was started from a 1 ml vial with approximately 500,000 cells stored in liquid nitrogen. Cells were propagated in 10 cm cell culture dishes by splitting every third day in a ratio of 1:3. Once confluent, fibroblasts were harvested to yield 3,000 cells/well in a 96 well plate. The remaining cells were distributed in 10 cm cell culture plates (600,000 cells/plate) for propagation. The plates were incubated overnight at 37° C. in an atmosphere with 95% humidity and 5% CO₂ to allow attachment of the cells to the culture plate. Plates were kept overnight in the cell culture incubator.

### Example B

Screening EPO-Mimetic Compounds in Fibroblasts from Friedreich's Ataxia (FRDA) Patients for Effect on Oxidative Phosphorylation.

Cells with well functioning electron transport chain should exhibit an increase in maximal oxygen consumption rate upon enhanced electron transport chain function. The effect of an EPO-mimetic compound on cellular oxidative phosphorylation was assessed via measurement of maximal oxygen consumption in growing cells. Treated cells exhibited a dose dependent increase in the maximal oxygen consumption capacity of their electron transport chain as measured with Seahorse instrument. Cells were grown as described in Example A, and assayed in the presence or absence of glycolysis inhibitors, such as 3BrPa, iodoacetate, fluoride, or 2-deoxyglucose, uncouplers such as FCCP or DNP, and various electron transport chain inhibitors such as rotenone and antimycin A. 1 IU of EPO-mimetic CNTO-530 increased maximal oxygen consumption by at least 30% and 2 IU increased the maximal oxygen by at least 80% as observed by mitochondrial uncoupling with FCCP and subsequent glycolysis inhibition with 2-deoxyglucose.

### Example C

Screening EPO-Mimetic Compounds in Fibroblasts from Friedreich's Ataxia (FRDA) Patients for Up-regulation of Electron Transport Chain Components

Treatment of FRDA cells grown as described in Example A with an EPO-mimetic compound may result in increased cellular electron transport chain protein content. Cells treated with EPO-mimetic CNTO-530 were analyzed by Western blot for electron transport chain protein and other regulatory protein amounts and correlated to untreated cells. An example of such proteins included but was not limited to frataxin, acconitase, and SOD. EPO-mimetic increased frataxin protein level in a dose dependent manner: 1 IU increased frataxin level by at least 100% and 2 IU by at least 200%. Increase in electron transport chain protein content was correlated to the improvement of mitochondrial function and oxidative phosphorylation.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of treating a mitochondrial disorder that is not a respiratory chain disorder, comprising:
administering a therapeutically effective amount of a composition comprising one or more EPO mimetic molecules or molecules capable of increasing the endogenous EPO or stimulating erythropoiesis, to an individual with a mitochondrial disorder that is not a respiratory chain disorder.

2. The method of claim 1, wherein the EPO mimetic molecule is a protein or a peptide, or a specified fragment or variant thereof.

3. The method of claim 1, wherein the EPO mimetic molecule is an EPO-mimetic antibody fusion protein.

4. The method of claim 3, wherein the EPO mimetic molecule is selected from CNTO-528 and CNTO-530.

5. The method of claim 4, wherein the EPO mimetic molecule increases the expression of frataxin by 50-300%.

6. The method of claim 1, wherein the composition comprises one or more molecules capable of increasing endogenous EPO or stimulating erythropoiesis.

7. The method of claim 6, wherein the molecule capable of increasing the endogenous EPO or stimulating erythropoiesis stabilizes the alpha subunit of hypoxia inducible factor (HIF-α).

8. The method of claim 6, wherein the molecule capable of increasing the endogenous EPO or stimulating erythropoiesis inhibits prolyl hydroxylation of HIF-α.

9. The method of claim 6, wherein the molecule capable of increasing the endogenous EPO or stimulating erythropoiesis is selected from FG-2216, FG-4539, FG-4592 and FG-6513.

10. The molecule of claim 6, wherein the molecule capable of increasing the endogenous EPO or stimulating erythropoiesis is Hematide™.

11. A method of treating Friedreich's ataxia, comprising:
administering a therapeutically effective amount of a composition comprising one or more EPO mimetic molecules or molecules capable of increasing endogenous EPO or stimulating erythropoiesis to an individual with Friedreich's ataxia.

12. The method of claim 11, wherein the EPO mimetic molecule is a protein or a peptide, or a specified fragment or variant thereof.

13. The method of claim 11, wherein the EPO mimetic molecule is a small molecule.

14. The method of claim 11, wherein the EPO mimetic molecule is an EPO-mimetic antibody fusion protein.

15. The method of claim 14, wherein the EPO mimetic molecule is selected from CNTO-528 and CNTO-530.

16. The method of claim 15, wherein the EPO mimetic molecule increases the expression of frataxin by 50-300%.

17. The method of claim 11, wherein the composition comprises a molecule capable of increasing the endogenous EPO or stimulating erythropoiesis.

18. The method of claim 17, wherein the molecule capable of increasing the endogenous EPO or stimulating erythropoiesis stabilizes the alpha subunit of hypoxia inducible factor (HIF-α).

19. The method of claim 17, wherein the molecule capable of increasing the endogenous EPO or stimulating erythropoiesis inhibits prolyl hydroxylation of HIF-α.

20. The method of claim 17, wherein the molecule capable of increasing the endogenous EPO or stimulating erythropoiesis is selected from FG-2216, FG-4539, FG-4592 and FG-6513.

21. The molecule of claim 17, wherein the molecule capable of increasing the endogenous EPO or stimulating erythropoiesis is Hematide™.

22. A method of treating Leigh's syndrome comprising:
administering a therapeutically effective amount of a composition comprising one or more EPO mimetic molecules or molecules capable of increasing the endogenous EPO or stimulating erythropoiesis to an individual with Leigh's syndrome.

23. The method of claim 22, wherein the EPO mimetic molecule is a protein or a peptide, or a specified fragment or variant thereof.

24. The method of claim 22, wherein the EPO mimetic molecule is a small molecule.

25. The method of claim 22, wherein the EPO mimetic molecule is an EPO-mimetic antibody fusion protein.

26. The method of claim 25, wherein the EPO mimetic molecule is selected from CNTO-528 and CNTO-530.

27. The method of claim 26, wherein the EPO mimetic molecule increases the expression of frataxin by 50-300%.

28. The method of claim 22, wherein the composition comprises a molecule capable of increasing the endogenous EPO or stimulating erythropoiesis.

29. The method of claim 28, wherein the molecule capable of increasing the endogenous EPO or stimulating erythropoiesis stabilizes the alpha subunit of hypoxia inducible factor (HIF-α).

30. The method of claim 28, wherein the molecule capable of increasing the endogenous EPO or stimulating erythropoiesis inhibits prolyl hydroxylation of HIF-α.

31. The method of claim 28, wherein the molecule capable of increasing the endogenous EPO or stimulating erythropoiesis is selected from FG-2216, FG-4539, FG-4592 and FG-6513.

32. The molecule of claim 28, wherein the molecule capable of increasing the endogenous EPO or stimulating erythropoiesis is Hematide™.

33. The method of claim 1, comprising:
administering a therapeutically effective amount of a composition comprising one or more EPO mimetic molecules or molecules capable of increasing the endogenous EPO or stimulating erythropoiesis to an individual with a mitochondrial disease selected from Parkinson's, Alzheimer's, amyotrophic lateral sclerosis (ALS) and Huntington's.

34. The method of claim 33, wherein the composition comprises an EPO mimetic molecule.

35. The method of claim 34, wherein the composition comprises CNTO-528 or CNTO-530.

36. The method of claim 35, wherein the EPO mimetic molecule increases the expression of frataxin by 50-300%.

37. The method of claim 33, wherein the composition comprises a molecule capable of increasing the endogenous EPO or stimulating erythropoiesis.

38. The method of claim 37, wherein the molecule capable of increasing the endogenous EPO is selected from FG-2216, FG-4539, FG-4592 and FG-6513.

39. The method of claim 37, wherein the molecule capable of increasing the endogenous EPO is Hematide™.

40. A method of treating a neurodegenerative disease caused by acquired mitochondrial dysfunction, comprising:
administering a therapeutically effective amount of a composition comprising one or more EPO mimetic molecules or molecules capable of increasing the endogenous EPO or stimulating erythropoiesis, to an individual with a neurodegenerative disease.

41. The method of claim 40, wherein the composition comprises an EPO mimetic molecule.

42. The method of claim 41, wherein the composition comprises CNTO-528 or CNTO-530.

43. The method of claim 42, wherein the EPO mimetic molecule increases the expression of frataxin by 50-300%.

44. The method of claim 40, wherein the composition comprises a molecule capable of increasing the endogenous EPO or stimulating erythropoiesis.

45. The method of claim 44, wherein the molecule capable of increasing the endogenous EPO is selected from FG-2216, FG-4539, FG-4592 and FG-6513.

46. The method of claim 44, wherein the molecule capable of increasing the endogenous EPO is Hematide™.
